(19) United States
(12) Patent Application Publication
(10) Pub. No.: US 2018/0363037 A1
(43) Pub. Date: Dec. 20, 2018

(54) DETECTION AND QUANTIFICATION OF NUCLEIC ACID MOLECULES ASSOCIATED WITH A SURFACE

(71) Applicants: LIFE TECHNOLOGIES CORPORATION, Carlsbad, CA (US); THERMO FISHER SCIENTIFIC GENEART GMBH, Regensburg (DE)

(72) Inventors: Korbinian HEIL, Munich (DE); Frank NOTKA, Regenstauf (DE); Kyle GEE, Springfield, OR (US); Jason DALLWIG, Eugene, OR (US); Phillip KUHN, Regensburg (DE); Schuyler CORRY, Eugene, OR (US)

(73) Assignees: LIFE TECHNOLOGIES CORPORATION, Carlsbad, CA (US); THERMO FISHER SCIENTIFIC GENEART GMBH, Regensburg (DE)

(21) Appl. No.: 15/781,899

(22) PCT Filed: Dec. 7, 2016
(86) PCT No.: PCT/US16/65325

§ 371 Date: Jun. 6, 2018

Related U.S. Application Data
(60) Provisional application No. 62/265,011, filed on Dec. 9, 2015.

(51) Int. Cl. C12Q 1/6834

(52) U.S. Cl. CPC C12Q 1/6834

(57) ABSTRACT

This disclosure relates to methods, compositions, and related apparatuses for detection and quantification of nucleic acid molecules associated with a solid surface. Methods can include detecting or quantifying at least one nucleic acid molecule associated with a surface by contacting the at least one nucleic acid molecule with a fluorophore, wherein the fluorophore emits increased fluorescence at a wavelength when in contact with nucleic acid molecule, and detecting or measuring fluorescence from the fluorophore at the wavelength. Compositions can comprise a fluorophore and a nucleic acid molecule, wherein the nucleic acid molecule is associated with a surface, and the fluorophore has the property of emitting increased fluorescence at a wavelength when in contact with a nucleic acid molecule.

### FIELD

This disclosure relates to the field of nucleic acid molecule detection and quantification.

### BACKGROUND

Detection and quantification of nucleic acid molecules has generally been performed using nucleic acid molecules in solution. Nucleic acid molecules are not solely provided and used in solution, however. Instead, they can be associated with a surface. It is desirable to detect or quantify nucleic acid molecules associated with a surface, for example, to provide quality control in synthetic processes such as microarray fabrication and other synthetic biochemical applications. It is also desirable to perform such detection or quantification on members of mixed populations, such as microarray spots or beads associated with different nucleic acid molecules.

Thus, there are needs for surface-associated nucleic acid molecule detection and quantification methods and related compositions and apparatuses. Provided herein are methods, uses, compositions, and apparatuses that can solve these needs and/or provide other benefits.

### SUMMARY

In some embodiments, the present disclosure provides a method of quantifying at least one (e.g., one, two, three, four, etc.) nucleic acid molecule (e.g., oligonucleotide) associated with a surface (e.g., slide, well, or bead), the method comprising: contacting the at least one nucleic acid molecule with a fluorophore, wherein the fluorophore emits increased fluorescence at a wavelength when in contact with nucleic acid molecule; and measuring fluorescence from the fluorophore at the wavelength.

Also provided herein is a method of detecting at least one nucleic acid molecule associated with a surface of a bead, the method comprising: contacting the at least one nucleic acid molecule with a fluorophore, wherein the fluorophore emits increased fluorescence at a wavelength when in contact with a nucleic acid molecule; and detecting fluorescence from the fluorophore at the wavelength.

Also provided herein is a noncovalent complex of a fluorophore and a nucleic acid molecule, wherein the nucleic acid molecule is associated with a surface of a bead, and the fluorophore has the property of emitting increased fluorescence at a wavelength when in contact with a nucleic acid molecule. In some embodiments, a noncovalent complex disclosed herein is for use in detecting or quantifying the nucleic acid molecule.

In some embodiments, the measured fluorescence is correlated to the mass of nucleic acid molecule associated with the surface. In some embodiments, the measured fluorescence is correlated to the molar quantity of nucleic acid molecule associated with the surface. In some embodiments, the measured fluorescence is compared to a reference. In some embodiments, the reference is a threshold value, a standard curve, or a value measured from a reference sample.

In some embodiments, the fluorophore is attached to a specific binding agent. In some embodiments, the specific binding agent comprises a nucleic acid molecule probe.

In some embodiments, the surface is associated with a plurality of nucleic acid molecules, with the nucleic acid molecules being in discrete locations on the surface.

In some embodiments, the surface is a surface of a slide or chip, optionally wherein the slide or chip is a microarray or silicon wafer. In some embodiments, the slide or chip comprises a semiconductor, glass, silanized glass, polyethyleneimine-coated glass, quartz, plastic, polystyrene, polypropylene, or polyethylene. In some embodiments, the slide or chip is nucleophilically or electrophilically derivatized. In some embodiments, the slide or chip comprises thiol, isothiocyanate, aldehyde, mercaptoalkyl, bromoacetamide, p-aminophenyl, epoxide, N-hydroxysuccinimidyl, imidoester, amino, cyanuric chloride, acrylic, carboxylic acid, maleimide, or disulfide functional groups.

In some embodiments, the surface is the surface of a bead. In some embodiments, the bead is a member of a mixed population of beads. In some embodiments, the bead has a size greater than or equal to about 0.05 μm, 0.1 μm, 0.2 μm, 0.3 μm, 0.5 μm, 1 μm, 2 μm, 3 μm, 5 μm, 10 μm, 20 μm, 30 μm, 40 μm, or 50 μm, and less than or equal to about 500 μm. In some embodiments, the bead has a size greater than or equal to about 0.1 μm and less than or equal to about 3 μm, 5 μm, 10 μm, 20 μm, 30 μm, 40 μm, 50 μm, 100 μm, 200 μm, 300 μm, 500 μm, 1 mm, or 2 mm. In some embodiments, the bead comprises plastic, ceramic, glass, polystyrene, methylstyrene, acrylic polymer, paramagnetic material, thoria sol, carbon graphite, titanium dioxide, latex, a cross-linked dextran, Sepharose, cellulose, nylon, cross-linked micelles, hydrogel, or polytetrafluoroethylene. In some embodiments, the bead is suspended in an aqueous medium. In some embodiments, the bead is dry. In some embodiments, the bead is suspended in an organic medium. In some embodiments, the bead is porous.

In some embodiments, the at least one nucleic acid molecule comprises a plurality of nucleic acid molecules with different sequences. In some embodiments, the at least one nucleic acid molecule consists essentially of nucleic acid molecules with a single sequence.

In some embodiments, the at least one nucleic acid molecule is attached to the surface through a noncovalent interaction. In some embodiments, the noncovalent interaction is between a hapten and a polypeptide or aptamer with affinity for the hapten.

In some embodiments, the at least one nucleic acid molecule is covalently linked to the surface. In some embodiments, the at least one nucleic acid molecule was covalently linked via reaction of a disulfide, thiol, amine, carboxyl, maleimide, phosphorothioate, aldehyde, alkylamino, acrylamide, or phosphoryl on the nucleic acid molecule with the surface.

In some embodiments, the at least one nucleic acid molecule was synthesized in situ. In some embodiments, the at least one nucleic acid molecule was synthesized in situ on a surface of a well of a multiwell plate. In some embodiments, the at least one nucleic acid molecule was synthesized in situ on a surface of a bead. In some embodiments, the in situ synthesis comprises enzymatic extension or ligation. In some embodiments, the in situ synthesis comprises addition of phosphoramidite nucleosides. In some embodiments, the method comprises synthesizing the at least one nucleic acid molecule on the surface before the contacting step.

In some embodiments, the at least one nucleic acid molecule comprises at least one nonstandard nucleotide. In some embodiments, the at least one nucleic acid molecule comprises at least one deoxyribonucleotide.

In some embodiments, the fluorophore emits increased fluorescence at the wavelength when in contact with a single-stranded nucleic acid molecule. In some embodiments, the at least one nucleic acid molecule comprises at least one single-stranded oligonucleotide.

In some embodiments, the fluorophore emits increased fluorescence at the wavelength when in contact with a double-stranded nucleic acid molecule. In some embodiments, the at least one nucleic acid molecule comprises at least one double-stranded nucleic acid molecule.

In some embodiments, the fluorophore comprises a cyanine dye, a phenanthridinium dye, a bisbenzimide dye, a bisbenzimidazole dye, an acridine dye, a chromomycinone dye, OLIGREEN®, PICOGREEN®, SYBR® Green, SYBR® Green II, SYBR® Gold, SYBR® Safe, CYQUANT® GR, DAPI, ethidium bromide, dihydroethidium, propidium iodide, hexidium iodide, QUANTIFLUOR® ssDNA dye, QUANTIFLUOR® dsDNA dye, a benzothiazolium dye, acridine orange, proflavine HCl, thiazole orange, oxazole yellow, chromomycin A3, 7-aminoactinomycin D, hydroxystilbamidine, HOECHST® 33258, HOECHST® 33342, thiazole orange tetramethylpropane diamine, thiazole orange tetramethyl diamine, ethidium propane diamine, or ethidium diethylene triamine.

In some embodiments, the at least one nucleic acid molecule comprises at least one protected moiety. In some embodiments, the at least one nucleic acid molecule comprises at least one unprotected nucleotide residue. In some embodiments, at least one unprotected nucleotide residue comprises an exocyclic amine.

In some embodiments, the detecting step is an in-line quality control step. In some embodiments, after detection, the at least one nucleic acid molecule is used in at least one downstream step. In some embodiments, a method disclosed herein further comprises reacting the at least one nucleic acid molecule after the detecting step. In some embodiments, the reacting comprises extending or ligating.

Also provided herein is an apparatus comprising a fluorescence excitation source, a fluorescence detector, and a noncovalent complex disclosed herein.

### BRIEF DESCRIPTION OF THE DRAWINGS

FIG. 1A-1B show microscope pictures (Zeiss) of polystyrene microspheres without oligonucleotides after treatment with QUANT-IT™ OLIGREEN® ss DNA Reagent in TE buffer. a) bright field b) fluorescence (excitation 480 nm).

FIG. 2A-2B show microscope pictures (Zeiss) of polystyrene microspheres coated with fully protected oligonucleotides after treatment with QUANT-IT™ OLIGREEN® ss DNA Reagent in TE buffer. a) bright field b) fluorescence (excitation 480 nm).

FIG. 3A-3B show microscope pictures (Zeiss) of polystyrene microspheres coated with oligonucleotides with protected exocyclic amines and deprotected phosphate backbone after treatment with QUANT-IT™ OLIGREEN® ss DNA Reagent in TE buffer. a) bright field b) fluorescence (excitation 480 nm).

FIG. 4 shows fluorescence microscope pictures (excitation 480 nm, Zeiss) of polystyrene microspheres coated with a 38mer oligonucleotide (left upper side) and 54mer oligonucleotide (right upper side) after treatment with QUANT-IT™ OLIGREEN® ss DNA Reagent in TE buffer. The corresponding intensity plots are shown under the microscope pictures.

FIG. 5A-5D show microscope pictures (Zeiss) of polystyrene microspheres coated with oligonucleotides after treatment with QUANT-IT™ OLIGREEN® ss DNA Reagent in TE buffer in a μ-multiwell plates in hydrated state. a) bright field (low magnification) b) fluorescence (low magnification, excitation 480 nm) c) bright field (high magnification) d) fluorescence (high magnification, excitation 480 nm).

FIG. 6A-6B show microscope pictures (Evos FL auto, GPF light cube) of polystyrene microspheres coated with oligonucleotides after treatment with QUANT-IT™ OLIGREEN® ss DNA Reagent hydrated in TE buffer in a μ-multiwell plates. a) fluorescence (low magnification, excitation 470 nm) b) fluorescence (high magnification, excitation 470 nm).

FIG. 7A-7C show microscope pictures (Zeiss) of polystyrene microspheres coated with oligonucleotides after treatment with QUANT-IT™ OLIGREEN® ss DNA Reagent in TE buffer in a μ-multiwell plates in hydrated state. a) bright field b) fluorescence (excitation 480 nm) c) fluorescence after washing with acetonitrile (excitation 480 nm).

### DETAILED DESCRIPTION

As used herein, “detect” means determining the presence or absence of an analyte such as an oligonucleotide and encompasses qualitative, semi-quantitative, and quantitative determinations. A quantitative determination gives a numerical value for the mass or molar quantity of the analyte, which will generally be subject to some degree of uncertainty due to typical sources of error. Molar quantity refers to the number of molecules, whether expressed as a literal number of molecules (e.g., 10¹⁴ molecules) or as a number or fraction of moles (e.g., 1 nanomole). A semi-quantitative determination gives at least an indication of the relative amount of the analyte, such as whether it is lower, approximately equal to, or higher than a threshold value or reference sample. In some embodiments, approximately equal to a value means within an order of magnitude. In some embodiments, approximately equal to means within or equal to five-fold. In some embodiments, approximately equal to means within or equal to two-fold. In some embodiments, approximately equal to means within or equal to 50%. In some embodiments, approximately equal to means within or equal to 34%. In some embodiments, approximately equal to means within or equal to 25%. In some embodiments, approximately equal to means within or equal to 20%. In some embodiments, approximately equal to means within or equal to 15%. In some embodiments, approximately equal to means within or equal to 10%. In some embodiments, approximately equal to means within or equal to 5%.

As used herein, “quantify” means determining the amount of an analyte, such as an oligonucleotide, and encompasses semi-quantitative and quantitative determinations.

As used herein, “determining an amount” means a quantitative determination.

As used herein, “nucleic acid molecule” generally refers to a molecule comprising linked nucleotides as subunits. A nucleic acid molecule can include nucleotides such as adenosine (A), cytosine (C), guanine (G), thymine (T), uracil (U), and nonstandard or modified nucleotides, which are discussed below. In some examples, a nucleic acid molecule comprises a deoxyribonucleic acid (DNA) segment, a ribonucleic acid (RNA) segment, or derivatives thereof, e.g., nucleic acid molecules comprising one or more phosphorothioate linkages, one or more peptide nucleic acid (PNA) nucleotides, one or more locked nucleic acid (LNA) nucleotides, one or more 2′-O-methylated sugars, etc. A nucleic acid molecule may be single-stranded or double stranded. In some embodiments, a nucleic acid molecule is an oligonucleotide.

As used herein, “at least one nucleic acid molecule” means one or more nucleic acid species equal to or shorter than about one kilobase. In the context of detection or quantification of surface-associated nucleic acid molecules, generally the at least one nucleic acid molecule will refer to a population of individual molecules. As used herein, nucleic acid molecules which differ only to the extent of point variations due to misincorporation or nonincorporation events (errors) during synthesis are considered to be the same species, i.e., they do not have different sequences, and the language “at least one nucleic acid molecule consists essentially of nucleic acid molecules with a single sequence” encompasses a population of nucleic acid molecules which differ only to the extent of point variations such as may result from misincorporation or nonincorporation events (errors) during synthesis. In contrast, a plurality of nucleic acid molecules with different sequences means that the nucleic acid molecules vary in sequence for a reason or reasons other than misincorporation or nonincorporation events during synthesis, e.g., they were synthesized in different reactions with different orders of addition of reagents, they were synthesized by extension along different template nucleic acids, or they were synthesized under mutagenic conditions or conditions that induce degenerate incorporation of nucleotides at one or more positions.

As used herein, “oligonucleotide” means an individual strand of linked nucleotides from about 2 to about 500 nucleotides in length. Oligonucleotides can be unhybridized or hybridized molecules (the latter being associated with another strand of linked nucleotides through base-pairing interactions). Oligonucleotides may be synthetic or may be made enzymatically. In some embodiments, an oligonucleotide is about 10 to 200 nucleotides in length. Oligonucleotides may contain ribonucleotide monomers (e.g., may be oligoribonucleotides), deoxyribonucleotide monomers, nonstandard nucleotide monomers, or combinations thereof. An oligonucleotide may be 10 to 20, 11 to 30, 31 to 40, 41 to 50, 51-60, 61 to 70, 71 to 80, 80 to 100, 100 to 150, or 150 to 200 nucleotides in length, for example.

As used herein, “fluorophore” means a chemical entity, including molecules and moieties, with at least one detectable excitation wavelength and at least one detectable emission wavelength different from the excitation wavelength, except that naturally occurring nucleobases; amino acid side chains; and protected, acylated, alkylated, etc., forms thereof are not considered fluorophores. Examples of fluorophores are discussed below.

As used herein “surface” means an interface of a solid-phase material, which may be macroscopic, mesoscopic, or microscopic, capable of covalent or non-covalent association, which association may be linker-mediated, with a nucleic acid molecule. Surfaces include interior surfaces of concave, semi-porous, porous, etc. materials as well as the exterior. Gels, e.g. made of agarose, polyacrylamide, etc., are considered solid. Items of various shapes may have surfaces. Exemplary items that have surfaces are beads, standard glass microscope slides, and multi-well plates (e.g., the wells of such plates have surfaces).

As used herein “bead” encompasses “microsphere” and refers to a solid particle having a globular or roughly spherical shape, which may be porous or non-porous. Non-porous surfaces may be present to increase surface area thus allowing for the association of increased number of surface bound molecules as compared to, for example, “smooth” surfaces.

As used herein “specific binding agent” means a chemical entity that recognizes a specific sequence, such that under appropriate conditions it binds nucleic acid molecules comprising the specific sequence with statistically significantly higher affinity than the affinity with which it binds an appropriate negative control molecule. For an oligodeoxynucleotide, an appropriate negative control molecule may be poly-dA, poly-dT, or random or bulk genomic DNA processed to have a size similar to the oligodeoxynucleotide. A specific sequence may contain one or more degenerate positions or be discontinuous. Many examples of sequence-specific DNA binding polypeptide domains, which are a type of specific binding agent, have recognition sequences with one or more degenerate positions or that are discontinuous. Another type of specific binding agent is an oligonucleotide probe, which recognizes complementary nucleotide sequences, as is well understood in the art. In some embodiments, an oligonucleotide probe is at least 80%, 90%, 95%, 98%, or 99% complementary to a recognition sequence. In some embodiments, an oligonucleotide probe is perfectly complementary to a recognition sequence.

As used herein “reacting” means any chemical process that results in covalent modification of a chemical entity.

As used herein “nonstandard nucleotide” means any nucleotide wherein the nucleobase is other than a standard base: adenine, guanine, cytosine, or (in DNA) thymine or (in RNA) uracil. Protected forms of standard bases such as are used in standard nucleic acid molecule synthesis methods are also considered standard.

As used herein “aqueous medium” means a liquid composition in which water makes up half or more than half of the liquid by mass.

As used herein “organic medium” means a liquid composition in which at least one organic solvent is present and water, if present, makes up less than half of the liquid by mass.

As used herein, “dry” means that an object is not immersed in liquid and its surface is in direct contact with a gas, such as air, N₂, O₂, argon, etc.

As used herein the terms “amplify”, “amplifying”, “amplification” and other related terms include producing multiple copies of an original biomolecule. In some embodiments, nucleic acid amplification produces multiple copies of an original polynucleotide (e.g., target polynucleotide), where the copies comprise a template sequence, or the copies comprise a sequence that is substantially identical to a template sequence.

A “template” refers to a polynucleotide sequence that comprises the polynucleotide to be amplified, flanked by primer hybridization sites. Thus, a “target template” comprises the target polynucleotide sequence flanked by hybridization sites for a 5′ primer (or the complement thereof) and a 3′ primer (or the complement thereof).

“Domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

“Identity” is measured by a score determined by comparing the sequences of the two biomolecules using the Bestfit program. Bestfit uses the local homology algorithm of Smith and Waterman, Advances in Applied Mathematics 2:482-489 (1981) to find the best segment of similarity between two sequences. When using Bestfit to determine whether a particular sequence is, for instance, 95% identical to a reference sequence, the parameters are set so that the percentage of identity is calculated over the full length of the reference amino acid sequence and that gaps in homology of up to 5% of the total number of residues in the reference sequence are allowed. “Complementarity” is measured as identity is except that the reverse complement of one of the sequences is used in the comparison.

As used herein the terms “hybridize”, “hybridizing”, “hybridization” and other related terms include hydrogen bonding between two different nucleic acids, or between two different regions of a nucleic acid, to form a duplex nucleic acid. Hybridization can comprise Watson-Crick or Hoogstein binding to form a duplex nucleic acid. The two different nucleic acids, or the two different regions of a nucleic acid, may be complementary, or partially complementary. The complementary base pairing can be the standard A-T or C-G base pairing, or can be other forms of base-pairing interactions. Duplex nucleic acids can include mismatched base-paired nucleotides. Complementary nucleic acid strands need not hybridize with each other across their entire length.

In some embodiments, conditions that are suitable for nucleic acid hybridization and/or for amplification conditions include parameters such as salts, buffers, pH, temperature, GC % content of the polynucleotide and primers, and/or time. For example, conditions suitable for hybridizing nucleic acids (e.g., polynucleotides and primers) can include hybridization solutions having sodium salts, such as NaCl, sodium citrate and/or sodium phosphate. In some embodiments, a hybridization solution can be a stringent hybridization solution which can include any combination of formamide (e.g., about 50%), 5×SSC (e.g., about 0.75 M NaCl and about 0.075 M sodium citrate), sodium phosphate (e.g., about 50 mM at about pH 6.8), sodium pyrophosphate (e.g., about 0.1%), 5×Denhardt's solution, SDS (e.g., about 0.1%), and/or dextran sulfate (e.g., about 10%). In some embodiments, hybridization and/or amplification can be conducted at a temperature range of about 45-55° C., or about 55-65° C., or about 65-75° C.

In some embodiments, hybridization or amplification conditions can be conducted at a pH range of about 5-10, or about pH 6-9, or about pH 6.5-8, or about pH 6.5-7.

Thermal melting temperature (Tₘ) for nucleic acids can be a temperature at which half of the nucleic acid strands are double-stranded and half are single-stranded under a defined condition. In some embodiments, a defined condition can include ionic strength and pH in an aqueous reaction condition. A defined condition can be modulated by altering the concentration of salts (e.g., sodium), temperature, pH, buffers, and/or formamide. Typically, the calculated thermal melting temperature can be at about 5-30° C. below the Tₘ, or about 5-25° C. below the Tₘ, or about 5-20° C. below the Tₘ, or about 5-15° C. below the Tₘ, or about 5-10° C. below the Tₘ. Methods for calculating a Tₘ are well known and can be found in Sambrook (1989 in “Molecular Cloning: A Laboratory Manual”, 2^{nd} edition, volumes 1-3; Wetmur 1966, J. Mol. Biol., 31:349-370; Wetmur 1991 Critical Reviews in Biochemistry and Molecular Biology, 26:227-259). Other sources for calculating a Tₘ for hybridizing or denaturing nucleic acids include OLIGOANALYZER® (from Integrated DNA Technologies) and Primer3 (distributed by the Whitehead Institute for Biomedical Research).

Provided herein are methods of detecting and/or quantifying at least one nucleic acid molecule (e.g., oligonucleotide) associated with a surface, as well as compositions for performing such methods. In some embodiments, such methods comprise contacting at least one nucleic acid molecule with a fluorophore, wherein the fluorophore emits increased fluorescence at a wavelength when in contact with the nucleic acid molecule; and detecting fluorescence from the fluorophore at the wavelength. In some embodiments, such methods comprise contacting at least one nucleic acid molecule with a fluorophore, wherein the fluorophore emits increased fluorescence at a wavelength when in contact with nucleic acid molecule; and measuring fluorescence from the fluorophore at the wavelength. The at least one nucleic acid molecule being detected or quantified is sometimes referred to as the analyte molecule; the use of the singular “analyte molecule” includes a population of molecules considered to have the same sequence, as discussed above, and also includes a population of heterogeneous nucleic acid molecules unless the context dictates otherwise.

Also provided herein are non-covalent complexes of one or more fluorophore and one or more nucleic acid molecule, wherein the nucleic acid molecules are associated with a surface, and the fluorophores have the property of emitting increased fluorescence (e.g., at a specified wavelength) when in contact with nucleic acid molecules. In some embodiments, non-covalent complexes of fluorophores and nucleic acid molecules may be used in detecting one or more nucleic acid molecule. In some embodiments, non-covalent complexes of fluorophores and nucleic acid molecules may be used for quantifying one or more nucleic acid molecule. Also provided herein is an apparatus comprising a fluorescence excitation source, a fluorescence detector, and a noncovalent complex of a fluorophore and a nucleic acid molecule, as described herein. In some embodiments, any of the foregoing is for use in determining the amount of the nucleic acid molecule present in a sample and/or localized one a surface.

In some embodiments, the fluorescence excitation source comprises a laser. In some embodiments, the fluorescence excitation source comprises a polychromatic light source. In some embodiments, the fluorescence excitation source comprises a mercury lamp. In some embodiments, the fluorescence excitation source comprises a monochromator. In some embodiments, the fluorescence excitation source comprises a filter. In some embodiments, the fluorescence detector comprises a photomultiplier tube. In some embodiments, the fluorescence detector comprises a charge-coupled device (CCD). In some embodiments, the fluorescence detector comprises a filter. In some embodiments, the fluorescence detector comprises a monochromator.

In some embodiments, the at least one nucleic acid molecule (e.g., oligonucleotide) has a length less than or equal to about one kilobase. In some embodiments, the at least one nucleic acid molecule has a length less than or equal to about 1,000 nucleotides (nts) (e.g., from about 10 nts to about 1,000 nts, from about 20 nts to about 1,000 nts, from about 30 nts to about 1,000 nts, from about 40 nts to about 1,000 nts, from about 50 nts to about 1,000 nts, from about 10 nts to about 500 nts, from about 20 nts to about 500 nts, from about 30 nts to about 500 nts, from about 40 nts to about 500 nts, from about 50 nts to about 500 nts, from about 20 nts to about 300 nts, from about 10 nts to about 50 nts, etc.).

In some embodiments, the at least one nucleic acid molecule has a length greater than or equal to about 6 nt (e.g., greater than or equal to about 6 nts, about 7 nts, about 8 nts, about 9 nts, about 10 nts, about 12 nts, about 15 nts, about 20 nts, about 30 nts, about 40 nts, about 50 nts, about 100 nts, about 200 nts, about 300 nts, about 400 nts, about 500 nts, etc.).

In some embodiments, the measured fluorescence is correlated to the mass of nucleic acid molecules associated with the surface. This can be so, for example, when multiple instances of the fluorophore bind along the length of the nucleic acid molecules, with more fluorophores binding when a greater mass of nucleic acid molecules is present. In some embodiments, the measured fluorescence is correlated to the molar quantity of nucleic acid molecules associated with the surface. This can be so, for example, when the fluorophore is covalently attached to a specific binding agent, such as a DNA binding domain or nucleic acid molecule probe that recognizes a sequence in the nucleic acid molecule, such that the number of bound fluorophores tracks the number of nucleic acid molecules in a length-independent manner. In some embodiments, the fluorophore is not covalently attached to a specific binding agent. In some embodiments, the fluorophore is not covalently attached to a chemical entity that comprises a nucleotide. In some embodiments, the fluorophore is not covalently attached to a nucleotide. In some embodiments, the fluorophore is not covalently attached to a nucleic acid molecule. In some embodiments, the fluorophore is not covalently attached to a DNA binding domain.

In some embodiments, the measured fluorescence is compared to a reference. In some embodiments, the reference is a threshold value. In some embodiments, the reference is a standard curve. In some embodiments, the reference is a value measured from a reference sample. In some embodiments, the reference sample has a known mass of nucleic acid molecules. In some embodiments, the reference sample has a known molar quantity of nucleic acid molecules. The measured fluorescence and reference can be expressed as fluorescence intensities measured in arbitrary units. The measured fluorescence and reference can also be expressed as a number of photons, a number of fluorophores, or a number or mass of analyte molecules, depending on the equipment used and the availability of information about the analyte and fluorophore such as quantum yield, concentration, binding affinity, etc.

In some embodiments, the at least one nucleic acid molecule comprises a plurality of nucleic acid molecules with different sequences. In some embodiments, such a plurality comprises a population of nucleic acid molecules with a combination of constant positions and degenerate positions. For example, the sequence ACTGACTGACTGN (SEQ ID NO: 1) where N means any one of the four standard deoxyribonucleotides has one degenerate and twelve constant positions. The sequence ACTGACTGACTGY (SEQ ID NO: 2) where Y means a pyrimidine base also has one degenerate and twelve constant positions. A population of nucleic acid molecules with a combination of constant positions and degenerate positions can be prepared by known methods, e.g., using a mixture of nucleotide monomers for the synthesis step(s) in which a degenerate position is to be added. In some embodiments, such a plurality comprises at least two nucleic acid molecules with unrelated sequences, e.g., which have less than or equal to 60% identity. In some embodiments, the plurality comprises at least two nucleic acid molecules which have less than or equal to 50% identity. In some embodiments, the plurality comprises at least two nucleic acid molecules which have less than or equal to 40% identity. In some embodiments, the plurality comprises at least two nucleic acid molecules which have less than or equal to 30% identity.

In some embodiments, the at least one nucleic acid molecule comprises or consists essentially of nucleic acid molecules with a single sequence. In some embodiments, the nucleic acid molecules with a single sequence were prepared in the same synthesis process. For the avoidance of doubt, if a method of this disclosure is performed on a spot in a microarray or, more generally, a particular region of a surface, and the nucleic acid molecules associated with that spot or region have the same sequence, then it would be true that the at least one nucleic acid molecule consists essentially of nucleic acid molecules with a single sequence—regardless of whether other nucleic acid molecules were present in other spots or in distinct regions of the surface.

In some embodiments, the surface is a surface of a slide. In some embodiments, the slide is a glass slide, such as a microscope slide. In some embodiments, the surface is a surface of a chip, such as a chip comprising a semiconductor, such as silicon. In some embodiments, the surface is a surface of a microarray. In some embodiments, the surface is a surface of a silicon wafer. In some embodiments, the slide or chip comprises a semiconductor, glass, quartz, or plastic. In some embodiments, the slide or chip comprises a plastic. In some embodiments, the plastic comprises polystyrene. In some embodiments, the plastic comprises polyethylene. In some embodiments, the plastic comprises polypropylene. In some embodiments, the slide or chip comprises a glass. In some embodiments, the glass comprises silanized glass. In some embodiments, the glass comprises polyethyleneimine-coated glass.

In some embodiments, the surface is associated with a plurality of nucleic acid molecules, with the nucleic acid molecules being in discrete locations on the surface. For example, in the context of a microarray, different nucleic acid molecules can be in different spots on the microarray. In the context of a multiwell plate, different nucleic acid molecules can be in different wells.

In some embodiments, the surface is nucleophilically or electrophilically derivatized. Such derivatization can facilitate coupling with an appropriately derivatized nucleic acid molecule. Commonly there will be residual derivatization following coupling. For purposes of this disclosure, residual derivatization and quenched or inactivated derivatization qualify as derivatization. In some embodiments, the surface comprises a reactive functional group. In some embodiments, the reactive functional group is a thiol. In some embodiments, the reactive functional group is an isothiocyanate. In some embodiments, the reactive functional group is an aldehyde. In some embodiments, the reactive functional group is a mercaptoalkyl. In some embodiments, the reactive functional group is a bromoacetamide. In some embodiments, the reactive functional group is a p-aminophenyl. In some embodiments, the reactive functional group is an epoxide. In some embodiments, the reactive functional group is a N-hydroxysuccinimidyl. In some embodiments, the reactive functional group is an imidoester. In some embodiments, the reactive functional group is an amino. In some embodiments, the reactive functional group is a cyanuric chloride. In some embodiments, the reactive functional group is an acrylic group. In some embodiments, the reactive functional group is a carboxylic acid. In some embodiments, the reactive functional group is a maleimide. In some embodiments, the reactive functional group is a disulfide.

In some embodiments, the at least one nucleic acid molecule is covalently linked to the surface. Such linkage can be formed via reaction of various groups on the nucleic acid molecule with an appropriate surface. In some embodiments, the at least one nucleic acid molecule is covalently linked via reaction of a reactive group on the nucleic acid molecule with the surface. In some embodiments, the reactive group comprised a disulfide. In some embodiments, the reactive group comprised a thiol. In some embodiments, the reactive group comprised an amine. In some embodiments, the reactive group comprised a carboxyl. In some embodiments, the reactive group comprised a maleimide. In some embodiments, the reactive group comprised a phosphorothioate. In some embodiments, the reactive group comprised an aldehyde. In some embodiments, the reactive group comprised an alkylamino. In some embodiments, the reactive group comprised an acrylamide. In some embodiments, the reactive group comprised a phosphoryl.

In some embodiments, the surface is the surface of a bead. In some embodiments, the at least one nucleic acid molecule was synthesized in situ on a surface of a bead. In some embodiments, the bead has a size greater than or equal to about 0.05 μm. In some embodiments, the bead has a size greater than or equal to about 0.1 μm. In some embodiments, the bead has a size greater than or equal to about 0.2 μm. In some embodiments, the bead has a size greater than or equal to about 0.3 μm. In some embodiments, the bead has a size greater than or equal to about 0.5 μm. In some embodiments, the bead has a size greater than or equal to about 1 μm. In some embodiments, the bead has a size greater than or equal to about 2 μm. In some embodiments, the bead has a size greater than or equal to about 3 μm. In some embodiments, the bead has a size greater than or equal to about 5 μm. In some embodiments, the bead has a size greater than or equal to about 10 μm. In some embodiments, the bead has a size greater than or equal to about 20 μm. In some embodiments, the bead has a size greater than or equal to about 30 μm. In some embodiments, the bead has a size greater than or equal to about 40 μm. In some embodiments, the bead has a size greater than or equal to about 50 μm.

In some embodiments, the bead has a size less than or equal to about 3 μm. In some embodiments, the bead has a size less than or equal to about 5 μm. In some embodiments, the bead has a size less than or equal to about 10 μm. In some embodiments, the bead has a size less than or equal to about 20 μm. In some embodiments, the bead has a size less than or equal to about 30 μm. In some embodiments, the bead has a size less than or equal to about 40 μm. In some embodiments, the bead has a size less than or equal to about 50 μm. In some embodiments, the bead has a size less than or equal to about 100 μm. In some embodiments, the bead has a size less than or equal to about 200 μm. In some embodiments, the bead has a size less than or equal to about 300 μm. In some embodiments, the bead has a size less than or equal to about 500 μm. In some embodiments, the bead has a size less than or equal to about 1 mm. In some embodiments, the bead has a size less than or equal to about 2 mm.

In some embodiments, the bead has a size ranging from about 0.05 μm to about 3 μm. In some embodiments, the bead has a size ranging from about 0.05 μm to about 5 μm. In some embodiments, the bead has a size ranging from about 0.05 μm to about 10 μm. In some embodiments, the bead has a size ranging from about 0.05 μm to about 20 μm. In some embodiments, the bead has a size ranging from about 0.05 μm to about 30 μm. In some embodiments, the bead has a size ranging from about 0.05 μm to about 40 μm. In some embodiments, the bead has a size ranging from about 0.05 μm to about 50 μm. In some embodiments, the bead has a size ranging from about 0.05 μm to about 100 μm. In some embodiments, the bead has a size ranging from about 0.05 μm to about 200 μm. In some embodiments, the bead has a size ranging from about 0.05 μm to about 300 μm. In some embodiments, the bead has a size ranging from about 0.05 μm to about 500 μm. In some embodiments, the bead has a size ranging from about 0.05 μm to about 1 mm. In some embodiments, the bead has a size ranging from about 0.05 μm to about 2 mm.

In some embodiments, the bead has a size ranging from about 0.05 μm to about 100 μm. In some embodiments, the bead has a size ranging from about 0.1 μm to about 2 mm. In some embodiments, the bead has a size ranging from about 0.2 μm to about 100 μm. In some embodiments, the bead has a size ranging from about 0.3 μm to about 50 μm. In some embodiments, the bead has a size ranging from about 0.5 μm to about 100 μm. In some embodiments, the bead has a size ranging from about 1 μm to about 1 mm. In some embodiments, the bead has a size ranging from about 2 μm to about 500 μm. In some embodiments, the bead has a size ranging from about 3 μm to about 200 μm. In some embodiments, the bead has a size ranging from about 5 μm to about 100 μm. In some embodiments, the bead has a size ranging from about 10 μm to about 50 μm. In some embodiments, the bead has a size ranging from about 10 μm to about 2 mm. In some embodiments, the bead has a size ranging from about 20 μm to about 40 μm. In some embodiments, the bead has a size ranging from about 20 μm to about 2 mm. In some embodiments, the bead has a size ranging from about 30 μm to about 35 μm. In some embodiments, the bead has a size ranging from about 30 μm to about 40 μm. In some embodiments, the bead has a size ranging from about 30 μm to about 2 mm. In some embodiments, the bead has a size ranging from about 40 μm to about 2 mm. In some embodiments, the bead has a size ranging from about 100 μm to about 2 mm.

In some embodiments, the bead comprises plastic. In some embodiments, the bead comprises ceramic. In some embodiments, the bead comprises glass. In some embodiments, the bead comprises polystyrene. In some embodiments, the bead comprises methylstyrene. In some embodiments, the bead comprises acrylic polymer. In some embodiments, the bead comprises paramagnetic material. In some embodiments, the bead comprises thoria sol. In some embodiments, the bead comprises carbon graphite. In some embodiments, the bead comprises titanium dioxide. In some embodiments, the bead comprises latex. In some embodiments, the bead comprises a cross-linked dextran. In some embodiments, the bead comprises Sepharose. In some embodiments, the bead comprises cellulose. In some embodiments, the bead comprises nylon. In some embodiments, the bead comprises cross-linked micelles. In some embodiments, the bead comprises a hydrogel. In some embodiments, the bead comprises polytetrafluoroethylene.

In some embodiments, the bead is suspended in an aqueous medium. In some embodiments, the bead is dry. In some embodiments, the bead is suspended in an organic medium.

In some embodiments, the organic medium comprises an aprotic solvent. In some embodiments, the organic medium comprises acetonitrile. In some embodiments, the organic medium comprises pyridine. In some embodiments, the organic medium comprises THF. In some embodiments, the organic medium comprises methylene chloride. In some embodiments, the organic medium comprises dimethyl formamide. In some embodiments, the organic medium comprises DMSO. In some embodiments, the organic medium comprises at least one solvent of the formula CH₃X wherein X is an electronegative moiety. In some embodiments, X is aprotic. In some embodiments, X is Cl, Br, NO₃, or CN.

In some embodiments, the bead is a member of a mixed population of beads. For example, a mixed population can comprise at least one bead associated with a nucleic acid molecule and at least one bead not associated with a nucleic acid molecule. Alternatively or in addition, a mixed population can comprise at least one bead associated with a first nucleic acid molecule and at least one bead associated with a second nucleic acid molecule, wherein the first and second nucleic acid molecules have sequences different from each other. In some embodiments, a mixed population comprises beads individually associated with at least about 3, 5, 10, 20, 30, 50, 100, 200, 300, 500, 1000, 2000, 3000, 5000, or 10000 nucleic acid molecules having different sequences. Methods of this disclosure can be performed on individual beads in mixed populations by obtaining spatially resolved fluorescence data.

In some embodiments, the medium comprises a buffer. In some embodiments, the buffer is Tris. In some embodiments, the buffer is HEPES. In some embodiments, the buffer is MOPS. In some embodiments, the pH is about 6 to 9. In some embodiments, the pH is about 6.5 to 8.5. In some embodiments, the pH is about 6.5 to 9. In some embodiments, the pH is about 7 to 9. In some embodiments, the pH is about 7 to 8. In some embodiments, the pH is about 7 to 8.5. In some embodiments, the pH is about 7.5 to 9. In some embodiments, the pH is about 7.5 to 8.5. In some embodiments, the medium comprises a chelator of a divalent cation. In some embodiments, the divalent cation is magnesium. In some embodiments, the chelator is EDTA.

In some embodiments, the bead is porous. In some embodiments, the surface area of the bead is at least about 2-fold higher than a non-porous sphere of equivalent mass and density. In some embodiments, the surface area of the bead is at least about 3-fold higher than a non-porous sphere of equivalent mass and density. In some embodiments, the surface area of the bead is at least about 4-fold higher than a non-porous sphere of equivalent mass and density. In some embodiments, the surface area of the bead is at least about 5-fold higher than a non-porous sphere of equivalent mass and density. In some embodiments, the surface area of the bead is at least about 7-fold higher than a non-porous sphere of equivalent mass and density. In some embodiments, the surface area of the bead is at least about 9-fold higher than a non-porous sphere of equivalent mass and density. In some embodiments, the surface area of the bead is less than or equal to about 12-fold higher than a non-porous sphere of equivalent mass and density. In some embodiments, the surface area of the bead is less than or equal to about 15-fold higher than a non-porous sphere of equivalent mass and density. In some embodiments, the surface area of the bead is less than or equal to about 20-fold higher than a non-porous sphere of equivalent mass and density.

Various surface materials, beads, slides, chips, and procedures for associating nucleic acid molecules with them such as the use of derivatization are discussed, e.g., in the following patents and publications: US 2013/0123121; U.S. Pat. No. 7,348,391; US 2014/0135233; US 2003/0143331; U.S. Pat. No. 8,507,197; U.S. Pat. No. 6,426,183; Fodor et al., Science 251:767 (1991); Beier et al., Nucl. Acids Res. 27:1970 (1999); U.S. Pat. No. 7,829,505.

In some embodiments, the at least one nucleic acid molecule is synthesized in situ. In some embodiments, the at least one nucleic acid molecule is synthesized in situ on a surface of a well of a multiwell plate. In some embodiments, the at least one nucleic acid molecule is synthesized in situ on a planar surface. In some embodiments, the at least one nucleic acid molecule is synthesized in situ on a surface of a bead. In some embodiments, the method comprises synthesizing the at least one nucleic acid molecule on the surface before the contacting step.

In some embodiments, the in situ synthesis comprises addition of phosphoramidite nucleosides. Phosphoramidite nucleosides are generally used in protected form. The protection can comprise protection of exocyclic amines (amines bonded to but not inside the purine or pyrimidine rings of nucleobases such as A, C, and G). In some embodiments, benzoyl (Bz) protecting groups are used. In some embodiments, acetyl (Ac) protecting groups are used. In some embodiments, phenoxyacetyl (PAC) protecting groups are used. In some embodiments, isobutyryl protecting groups are used. The protection can comprise protection of the phosphate backbone. In some embodiments, cyanoethyl protecting groups are used. The protection can comprise protection of the 5′ hydroxyl. In some embodiments, a DMT (4,4′-dimethoxytrityl) protecting group is used. In some embodiments relating to a 2′ hydroxyl-bearing oligonucleotides (e.g., RNA), the protection can comprise protection of the 2′ hydroxyl. In some embodiments, a TBDMS (t-butyldimethylsilyl) protecting group is used. In some embodiments, a TOM (tri-iso-propylsilyloxymethyl) protecting group is used. In some embodiments, at least one exocyclic amine is unprotected. In some embodiments, at least one phosphodiester linkage is unprotected. Nucleic acid molecule synthesis and protection strategy is extensively discussed in the literature, including methods for synthesis of oligonucleotides involving surfaces. See, e.g., U.S. Pat. No. 6,028,189; U.S. Pat. No. 8,461,317; US 2014/0350235; Gryaznov, S. M.; Letsinger, R. L. (1991). “Synthesis of oligonucleotides via monomers with unprotected bases.” J. Amer. Chem. Soc. 113 (15): 5876-5877. doi:10.1021/ja00015a059; Reddy, M. P.; Hanna, N. B.; Farooqui, F. (1997). “Ultrafast Cleavage and Deprotection of Oligonucleotides Synthesis and Use of CAc Derivatives.” Nucleosides & Nucleotides 16: 1589-1598. doi:10.1080/07328319708006236; McMinn, D. (1997). “Synthesis of oligonucleotides containing 3′-alkyl amines using N-isobutyryl protected deoxyadenosine phosphoramidite.” Tetrahedron Lett. 38: 3123. doi:10.1016/S0040-4039(97)00568-6; Schulhof, J. C.; Molko, D.; Teoule, R. (1987). “The final deprotection step in nucleic acid molecule synthesis is reduced to a mild and rapid ammonia treatment by using labile base-protecting groups.” Nucleic Acids Res. 15 (2): 397-416. doi:10.1093/nar/15.2.397. PMC 340442. PMID 3822812; Zhu, Q. (2001). “Observation and elimination of N-acetylation of oligonucleotides prepared using fast-deprotecting phosphoramidites and ultra-mild deprotection.” Bioorg. & Med. Chem. Lett. 11: 1105. doi:10.1016/50960-894X(01)00161-5; McBride, L. J.; Kierzek, R.; Beaucage, S. L.; Caruthers, M. H. (1986). “Nucleotide chemistry. 16. Amidine protecting groups for oligonucleotide synthesis.” J. Amer. Chem. Soc. 108: 2040. doi:10.1021/ja00268a052; Sinha, N. D.; Biernat, J.; McManus, J.; Koester, H. (1984). “Polymer support oligonucleotide synthesis. XVIII: use of β-cyanoethyl-N,N-dialkylamino-/N-morpholino phosphoramidite of deoxynucleosides for the synthesis of DNA fragments simplifying deprotection and isolation of the final product.” Nucleic Acids Res 12 (11): 4539-4557. doi:10.1093/nar/12.11.4539. PMC 318857. PMID 6547529; Guzaev, A. P.; Manoharan, M. (2001). “Phosphoramidite Coupling to Oligonucleotides Bearing Unprotected Internucleosidic Phosphate Moieties.” J. Org. Chem. 66 (5): 1798-1804. doi:10.1021/jo001591e. PMID 11262130; Ogilvie, K. K.; Theriault, N.; Sadana, K. L. (1977). “Synthesis of oligoribonucleotides.” J. Amer. Chem. Soc. 99 (23): 7741-7743. doi:10.1021/ja00465a073; Usman, N.; Ogilvie, K. K.; Jiang, M. Y.; Cedergren, R. J. (1987). “The automated chemical synthesis of long oligoribuncleotides using 2′-O-silylated ribonucleoside 3′-O-phosphoramidites on a controlled-pore glass support: synthesis of a 43-nucleotide sequence similar to the 3′-half molecule of an Escherichia coli formylmethionine tRNA.” J. Amer. Chem. Soc. 109 (25): 7845-7854. doi:10.1021/ja00259a037; Usman, N.; Pon, R. T.; Ogilvie, K. K. (1985). “Preparation of ribonucleoside 3′-O-phosphoramidites and their application to the automated solid phase synthesis of oligonucleotides.” Tetrahedron Lett. 26 (38): 4567-4570. doi:10.1016/S0040-4039(00)98753-7; Scaringe, S. A.; Francklyn, C.; Usman, N. (1990). “Chemical synthesis of biologically active oligoribonucleotides using β-cyanoethyl protected ribonucleoside phosphoramidites.” Nucl. Acids Res. 18 (18): 5433-5441. doi:10.1093/nar/18.18.5433; Pitsch, S.; Weiss, P. A.; Wu, X.; Ackermann, D.; Honegger, T. (1999). “Fast and reliable automated synthesis of RNA and partially 2′-O-protected precursors (“caged RNA”) based on two novel, orthogonal 2′-O-protecting groups.” Helv. Chim. Acta 82 (10): 1753-1761. doi:10.1002/(SICI)1522-2675(19991006)82:10<1753::AID-HLCA1753>3.0. CO;2-Y; Pitsch, S.; Weiss, P. A.; Jenny, L.; Stutz, A.; Wu, X. (2001). “Reliable chemical synthesis of oligoribonucleotides (RNA) with 2′-O-[(triisopropylsilyl)oxy]methyl(2′-O-tom)-protected phosphoramidites.” Helv. Chim. Acta 84 (12): 3773-3795. doi:10.1002/1522-2675(20011219)84:12<3773::AID-HLCA3773>3.0.CO;2-E.

In some embodiments, the in situ synthesis comprises enzymatic extension. In some embodiments, the in situ synthesis comprises enzymatic ligation. Ligase and polymerase enzymes are commercially available. In some embodiments, the enzymatic extension or ligation is performed subsequent to initial chemical synthesis (e.g., addition of phosphoramidite nucleosides followed by deprotection).

In some embodiments, the at least one nucleic acid molecule is attached to the surface through a noncovalent interaction. In some embodiments, the hapten comprises biotin. In some embodiments, the hapten comprises digoxigenin. In some embodiments, the noncovalent interaction is between a hapten and a polypeptide with affinity for the hapten. In some embodiments, the polypeptide comprises an antibody, which does not necessarily contain domains or sequences nonessential for binding (e.g., the sequences omitted from Fab fragments). In some embodiments, the polypeptide comprises avidin or streptavidin. In some embodiments, the noncovalent interaction is between a hapten and an aptamer with affinity for the hapten.

In some embodiments, the at least one nucleic acid molecule comprises at least one nonstandard nucleotide. Some examples of nonstandard nucleotides include, but are not limited to, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. In some embodiments, the nonstandard nucleotide comprises, e.g., 5-methylcytosine, 5-bromouracil, uracil, 5,6-dihydrouracil, ribothymine, 7-methylguanine, hypoxanthine, pseudouridine, inosine, or xanthine. Uracil in DNA is also a nonstandard base. In some embodiments, the nonstandard base is incorporated during synthesis. In some embodiments, the nonstandard base is formed by chemical or enzymatic modification of a base in a nucleic acid molecule.

In some embodiments, the at least one nucleic acid molecule comprises at least one deoxyribonucleotide. In some embodiments, the at least one nucleic acid molecule comprises at least one ribonucleotide. In some embodiments, the at least one nucleic acid molecule comprises at least one bicyclic nucleoside analog, such as an LNA (locked nucleic acid) unit. In some embodiments, the at least one nucleic acid molecule comprises at least one phosphorothioate linkage. In some embodiments, the at least one nucleic acid molecule comprises at least one 2′-methoxy. In some embodiments, the at least one nucleic acid molecule comprises at least one peptide nucleic acid (PNA) unit.

In some embodiments, the at least one nucleic acid molecule comprises at least one single-stranded nucleic acid molecule. In some embodiments, the at least one nucleic acid molecule comprises at least one double-stranded nucleic acid molecule.

In some embodiments, the fluorophore emits increased fluorescence at the wavelength when in contact with single-stranded nucleic acid molecules. In some embodiments, the fluorophore emits increased fluorescence at the wavelength when in contact with double-stranded nucleic acid molecules.

In some embodiments, the fluorophore comprises a cyanine dye. In some embodiments, the fluorophore comprises a phenanthridinium dye. In some embodiments, the fluorophore comprises a bisbenzimide dye. In some embodiments, the fluorophore comprises a bisbenzimidazole dye. In some embodiments, the fluorophore comprises an acridine dye. In some embodiments, the fluorophore comprises a chromomycinone dye. In some embodiments, the fluorophore comprises OLIGREEN® (Thermo Fisher Scientific, Inc.). In some embodiments, the fluorophore comprises PICOGREEN® (Thermo Fisher Scientific, Inc.). In some embodiments, the fluorophore comprises SYBR® Green (Thermo Fisher Scientific, Inc.) (N′,N′-dimethyl-N-[4-[(E)-(3-methyl-1,3-benzothiazol-2-ylidene)methyl]-1-phenylquinolin-1-ium-2-yl]-N-propylpropane-1,3-diamine). In some embodiments, the fluorophore comprises SYBR® Green II (Thermo Fisher Scientific, Inc.). In some embodiments, the fluorophore comprises SYBR® Gold (Thermo Fisher Scientific, Inc.). In some embodiments, the fluorophore comprises SYBR® Safe DNA gel stain (Thermo Fisher Scientific, Inc.). In some embodiments, the fluorophore comprises CYQUANT® GR dye (Thermo Fisher Scientific, Inc.). In some embodiments, the fluorophore comprises EVAGREEN® (Biotium, Inc.). In some embodiments, the fluorophore comprises DAPI (4′,6-diamidino-2-phenylindole). In some embodiments, the fluorophore comprises ethidium bromide. In some embodiments, the fluorophore comprises ethidium homodimer-1. In some embodiments, the fluorophore comprises ethidium homodimer-2. In some embodiments, the fluorophore comprises propidium iodide. In some embodiments, the fluorophore comprises dihydroethidium. In some embodiments, the fluorophore comprises hexidium iodide. In some embodiments, the fluorophore comprises DAPI. In some embodiments, the fluorophore comprises QUANTIFLUOR® ssDNA dye (Promega Corp.). In some embodiments, the fluorophore comprises QUANTIFLUOR® dsDNA dye (Promega Corp.). In some embodiments, the fluorophore comprises a benzothiazolium dye. In some embodiments, the fluorophore comprises acridine orange. In some embodiments, the fluorophore comprises proflavine HCl. In some embodiments, the fluorophore comprises thiazole orange. In some embodiments, the fluorophore comprises oxazole yellow. In some embodiments, the fluorophore comprises chromomycin A3. In some embodiments, the fluorophore comprises 7-aminoactinomycin D. In some embodiments, the fluorophore comprises hydroxystilbamidine. In some embodiments, the fluorophore comprises HOECHST® 33258. In some embodiments, the fluorophore comprises HOECHST® 33342. In some embodiments, the fluorophore comprises thiazole orange tetramethylpropane diamine. In some embodiments, the fluorophore comprises thiazole orange tetramethyl diamine. In some embodiments, the fluorophore comprises ethidium propane diamine. In some embodiments, the fluorophore comprises ethidium diethylene triamine. In some embodiments, the fluorophore comprises TOTO-1. In some embodiments, the fluorophore comprises TO-PRO-1. In some embodiments, the fluorophore comprises POPO-1. In some embodiments, the fluorophore comprises BOBO-1. In some embodiments, the fluorophore comprises YOYO-1. In some embodiments, the fluorophore comprises JOJO-1. In some embodiments, the fluorophore comprises POPO-3. In some embodiments, the fluorophore comprises LOLO-1. In some embodiments, the fluorophore comprises BOBO-3. In some embodiments, the fluorophore comprises YOYO-3. In some embodiments, the fluorophore comprises TOTO-3. In some embodiments, the fluorophore comprises PO-PRO-1. In some embodiments, the fluorophore comprises YO-PRO-1. In some embodiments, the fluorophore comprises JO-PRO-1. In some embodiments, the fluorophore comprises PO-PRO-3. In some embodiments, the fluorophore comprises YO-PRO-3. In some embodiments, the fluorophore comprises TO-PRO-3. In some embodiments, the fluorophore comprises TO-PRO-5. In some embodiments, the fluorophore comprises SYTOX® Blue. In some embodiments, the fluorophore comprises SYTOX® Green. In some embodiments, the fluorophore comprises SYTOX® Orange. In some embodiments, the fluorophore comprises SYTOX® Red. In some embodiments, the fluorophore comprises at least one of SYTO® 40, 41, 42, or 45. In some embodiments, the fluorophore comprises at least one of SYTO® 9, 10, 11, 12, 13, 14, 16, 21, 24, or 25. In some embodiments, the fluorophore comprises SYTO® RNASelect. In some embodiments, the fluorophore comprises SYTO® BC. In some embodiments, the fluorophore comprises at least one of SYTO® 80, 81, 82, 83, 84, or 85. In some embodiments, the fluorophore comprises at least one of SYTO® 17, 59, 60, 61, 62, 63, or 64. In some embodiments, the fluorophore comprises bis-(6-chloro-2-methoxy-9-acridinyl)spermine. In some embodiments, the fluorophore comprises quinacrine. In some embodiments, the fluorophore comprises 9-amino-6-chloro-2-methoxyacridine. In some embodiments, the fluorophore comprises LDS 751. In some embodiments, the fluorophore comprises daunomycin. In some embodiments, the fluorophore comprises mithramycin A. In some embodiments, the fluorophore comprises olivomycin. In some embodiments, the fluorophore comprises chromomycin A3.

Fluorophores that may be used in the practice of the invention may bind preferentially to DNA over RNA, preferentially to RNA over DNA, and/or preferentially to single-stranded (ss) nucleic acids over double-stranded (ds) nucleic acids. Thus, fluorophores that may be used in the practice of the invention may binding preferentially to ssDNA over dsDNA.

Further, fluorophores that may be used in the practice of the invention may be “non-destructive” in the sense that they do not interfere with “downstream” uses of detected/quantified nucleic acid molecules. Thus, for example, a fluorophore may be used to quantified nucleic acid molecule covalently bound to a bead, the flurorophore may then be separated from the nucleic acid molecule, and the nucleic acid molecule may then be used in a process (e.g., as a PCR primer).

In some embodiments, the detecting step is an in-line quality control step. Thus, the method may be used as part of a larger scheme, such as a scheme for synthesizing nucleic acid molecules for use in the preparation of polynucleotides such as ORFs, synthetic genes, vectors, minichromosomes, viral genomes, YACs, BACs, extrachromosomal elements, plasmids, cosmids, fosmids, chromosomes, genomes, etc. The detecting step can qualitatively, semi-quantitatively, or quantitatively indicate the success or yield of a first reaction before proceeding to a second reaction. In some embodiments, a second reaction is performed, such as a ligation or extension, if the results of the detection method so indicate, e.g., if the results qualitatively indicate the presence of nucleic acid molecule, semi-quantitatively indicate that the amount of nucleic acid molecule exceeds a threshold, or quantitatively indicate an amount suitable for use in the second reaction. In some embodiments, the method is non-destructive. Put another way, the method is performed on a nucleic acid molecule preparation that after the method remains suitable for use in a subsequent reaction. In some embodiments, after detection, the nucleic acid molecules are used in at least one downstream step. In some embodiments, the at least one downstream step comprises an extension reaction.

In some embodiments, the extension reaction is a nucleic acid amplification reaction. In some embodiments, the amplification reaction includes a cycled amplification reaction, such as a polymerase chain reaction (PCR) (U.S. Pat. Nos. 4,683,195 and 4,683,202 both granted to Mullis). In some embodiments, the nucleic acid amplification reaction includes an isothermal reaction, such as an isothermal self-sustained sequence reaction (Kwoh 1989 Proceedings National Academy of Science USA 86:1173-1177; WO 1988/10315; and U.S. Pat. Nos. 5,409,818, 5,399,491, and 5,194,370), or a recombinase polymerase amplification (RPA) (U.S. Pat. No. 5,223,414 to Zarling, U.S. Pat. Nos. 5,273,881 and 5,670,316 both to Sena, and U.S. Pat. Nos. 7,270,981, 7,399,590, 7,435,561, 7,666,598, 7,763,427, 8,017,339, 8,030,000, 8,062,850, and 8,071,308).

PCR is a DNA synthesis reaction in which the reaction mixture is subjected to at least two complete reaction cycles, each reaction cycle comprising a denaturation period and at least one annealing and/or extension period, resulting if successful in synthesis of copies of a nucleic acid template in at least the initial cycles, and copies of the copies in at least the later cycles, generally resulting in geometric amplification of the template. In PCR, a pair of primers are provided that bind at each end of a target region, on opposite strands such that they each prime synthesis toward the other primer. The reaction is thermocycled so as to drive denaturation of the substrate in a high temperature step, annealing of the primers at a lower temperature step, and extension at a temperature which may be but is not necessarily higher than that of the annealing step. Geometric amplification occurs because the products of one cycle can serve as template in the next cycle.

An embodiment of isothermal self-sustained sequence reaction, also sometimes referred to as transcription-mediated amplification or TMA, involves synthesizing single-stranded RNA, single-stranded DNA and double-stranded DNA. The single-stranded RNA is a first template for a first primer, the single-stranded DNA is a second template for a second primer, and the double stranded DNA is a third template for synthesis of a plurality of copies of the first template. A sequence of the first primer or the second primer is complementary to a sequence of a target nucleic acid and a sequence of the first primer or the second primer is homologous to a sequence of the target nucleic acid. In an embodiment of an isothermal self-sustained sequence reaction, a first cDNA strand is synthesized by extension of the first primer along the target by an enzyme with RNA-dependent DNA polymerase activity, such as a reverse transcriptase. The first primer comprises a polymerase binding sequence (PBS) such as a PBS for a DNA-dependent RNA polymerase, such as T7, T3, or SP6 RNA polymerase. The first primer comprising a PBS is sometimes referred to as a promoter-primer. The first cDNA strand is rendered single-stranded, such as by denaturation or by degradation of the RNA, such as by an RNase H. The second primer then anneals to the first cDNA strand and is extended to form a second cDNA strand by a DNA polymerase activity. Forming the second cDNA strand renders the cDNA double-stranded, including the PBS. RNA can then be synthesized from the cDNA, which comprises the PBS, by a DNA-dependent RNA polymerase, such as T7, T3, or SP6 RNA polymerase, thereby providing a template for further events (extension of the first primer, rendering the product single-stranded, extension of the second primer, and RNA synthesis). Geometric amplification occurs because the RNA product can subsequently serve as a template and also because RNA products can be generated repeatedly from a cDNA comprising the PBS. An embodiment of RPA can be performed isothermally and employs a recombinase to promote strand invasion of a double-stranded template by forward and reverse primers. The 3′ ends of the primers are extended, displacing template strands at least in part. Subsequent strand invasion/annealing events, including to previously produced extension products, occur and are followed by extension, resulting in amplification. In some embodiments, recombinase activity is supported by the presence of one or more recombinase accessory proteins, such as a recombinase loading protein and/or single-stranded binding protein. In some embodiments isothermal amplification conditions comprise a nucleic acid amplification reaction mixture that is subjected to a temperature variation which is constrained within a limited range during at least some portion of the amplification, including for example a temperature variation is within about 20° C., or about 10° C., or about 5° C., or about 1-5° C., or about 0.1-1° C., or less than about 0.1° C. In some embodiments, an isothermal nucleic acid amplification reaction can be conducted at about 15-30° C., or about 30-45° C., or about 45-60° C., or about 60-75° C., or about 75-90° C., or about 90-93° C., or about 93-99° C.

In some embodiments, the at least one downstream step comprises a ligation reaction. In some embodiments, the at least one downstream step comprises a derivatization reaction. In some embodiments, the derivatization reaction comprises attachment of a label. In some embodiments, the label can include compounds that are fluorophores, chromophores, radioisotopes, haptens, affinity tags, atoms or enzymes. In some embodiments, the label comprises a moiety not typically present in naturally occurring nucleotides. For example, the label can include fluorescent, luminescent or radioactive moieties. In some embodiments, the label comprises a fluorophore, such as fluorescein, FITC, a cyanine dye, or any of the fluorophores discussed above. In some embodiments, the label comprises a hapten, such as biotin or digoxigenin. In some embodiments, the label comprises an enzyme, such as horse radish peroxidase or luciferase.

In some embodiments, after detection, the nucleic acid molecules are used in at least one downstream step. In some embodiments, a method according to this disclosure further comprises reacting the plurality of nucleic acid molecules after the detecting step. In some embodiments, reacting comprises extending or ligating.

### EXAMPLES

The following examples are provided to illustrate certain disclosed embodiments and are not to be construed as limiting the scope of this disclosure in any way.

### Example 1. Staining of Oligonucleotide Microspheres Using a Fluorogenic Dye

Provided were 30 μm polystyrene microspheres (Custom Primer Support 80S (dT, GE Healthcare)) with covalent bound fully protected oligonucleotide (5′-CGTGTCTTGTCCAGAGCTCTCATAGAACAACCGTCCCT-3′) (SEQ ID NO: 3), prepared using an ABI392 DNA Synthesizer (Applied Biosystems) according to standard procedures involving acetonitrile (Anhydrous for DNA Synthesis, Fisher Bioreagents), dicyanoimidazole as activator (Sigma Aldrich), DMT-dA(bz)phosphoramidite (Sigma Aldrich), DMT-dG(ib) phosphoramidite (Sigma Aldrich), DMT-dC(bz) phosphoramidite (Sigma Aldrich), DMT-dT phosphoramidite (Sigma Aldrich), CAP A (Sigma Aldrich), CAP B (Sigma Aldrich), oxidizer 0.1 M (Sigma Aldrich), TCA deblock (Sigma Aldrich).

The oligonucleotide-coated microspheres were dried and directly used for staining.

In order to produce oligonucleotide coated microspheres with protected exocyclic amines and deprotected phosphate backbone, the microspheres were rinsed with 50% triethylamine in acetonitrile and subsequently washed with acetonitrile to remove the cyanoethyl protecting groups (See Capaldi, D. C.; Gaus, H.; Krotz, A. H.; et al. (2003). “Synthesis of High-Quality Antisense Drugs. Addition of Acrylonitrile to Phosphorothioate Oligonucleotides: Adduct Characterization and Avoidance”. Org. Proc. Res. & Development 7 (6): 832-838).

Polystyrene microspheres (Custom Primer Support 80S (dT, GE Healthcare)) without oligonucleotides were used as a control.

The microspheres were flushed into weir-filter chips (slit height 10 μm, material Zeonor, microfluidic ChipShop) to enable rinsing with staining solution while being optically accessible. The microspheres were treated with a working solution of QUANT-IT OLIGREEN® ss DNA Reagent (Thermo Fisher Scientific) prepared according to the manufacturer's instructions in TE buffer.

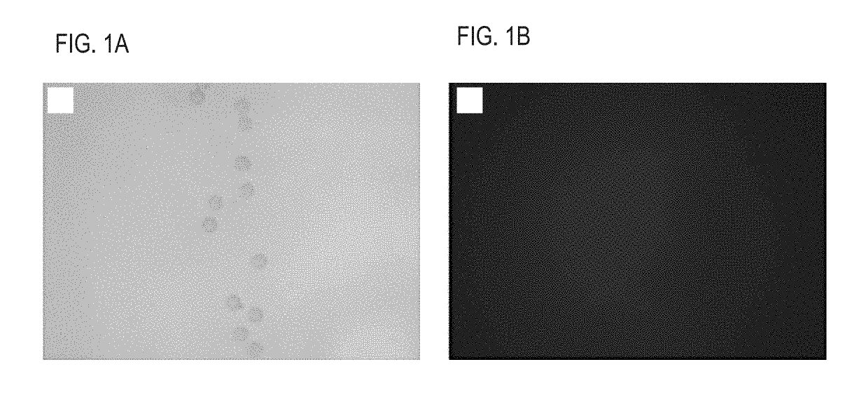

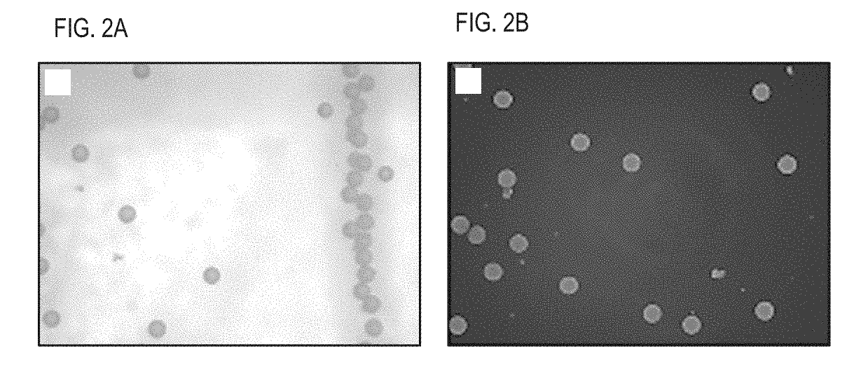

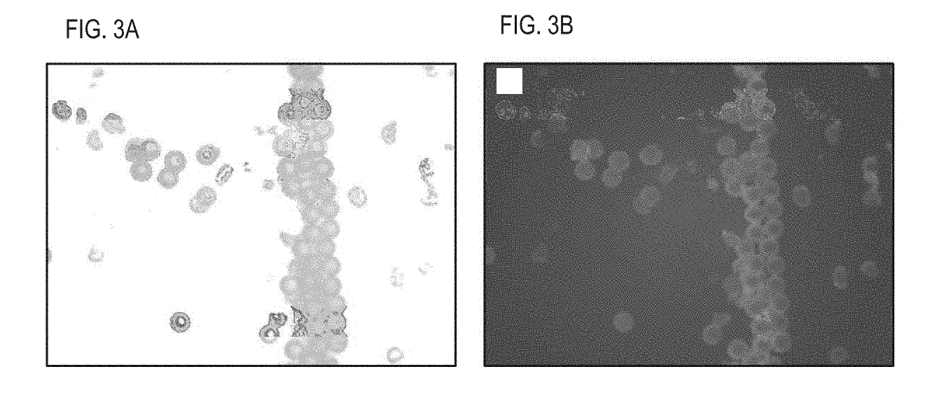

The microspheres were excited with 480 nm light and fluorescence was tracked using a Zeiss Fluorescence Microscope. No fluorescence signal was observed for the polystyrene beads without attached oligonucleotides (see FIG. 1A-1B). The microspheres coated with fully protected oligonucleotides (see FIG. 2A-2B) together with the microspheres coated with oligonucleotides with protected exocyclic amines and deprotected phosphate backbone (FIG. 3A-3B) showed a strong fluorescence signal.

### Example 2. Semi Quantitative Fluorescence Measurement of OLIGREEN®-Stained Oligonucleotide Microspheres

Provided was an oligonucleotide sequence (38 bases, 5′-CGTGTCTTGTCCAGAGCTCTCATAGAACAACCGTCCCT-3′) (SEQ ID NO: 3), covalently bound to 30 μm polystyrene microspheres (Custom Primer Support 80S (dT, GE Healthcare)) prepared with an ABI392 DNA Synthesizer (Applied Biosystems) essentially as in Example 1. Also provided was an oligonucleotide sequence (54 bases, 5′-TTGAATAATTCGTCGTGGCATACAGCCGGGGTTGCTGTAAAACCCCTAACT AGG-3′) (SEQ ID NO: 4), covalently bound to 30 μm polystyrene microspheres (Custom Primer Support 80S (dT, GE Healthcare)) prepared with an ABI392 DNA Synthesizer (Applied Biosystems) essentially as in Example 1.

The 30 μm polystyrene microspheres coated with fully protected oligonucleotide were dried and used directly for staining.

The microspheres were flushed into weir-filter chips (slit height 10 μm, material Zeonor, microfluidic ChipShop) to facilitate rinsing with staining solution while being optically accessible. The microspheres were treated with QUANT-IT OLIGREEN® ss DNA Reagent (Thermo Fisher Scientific) in TE buffer as in Example 1.

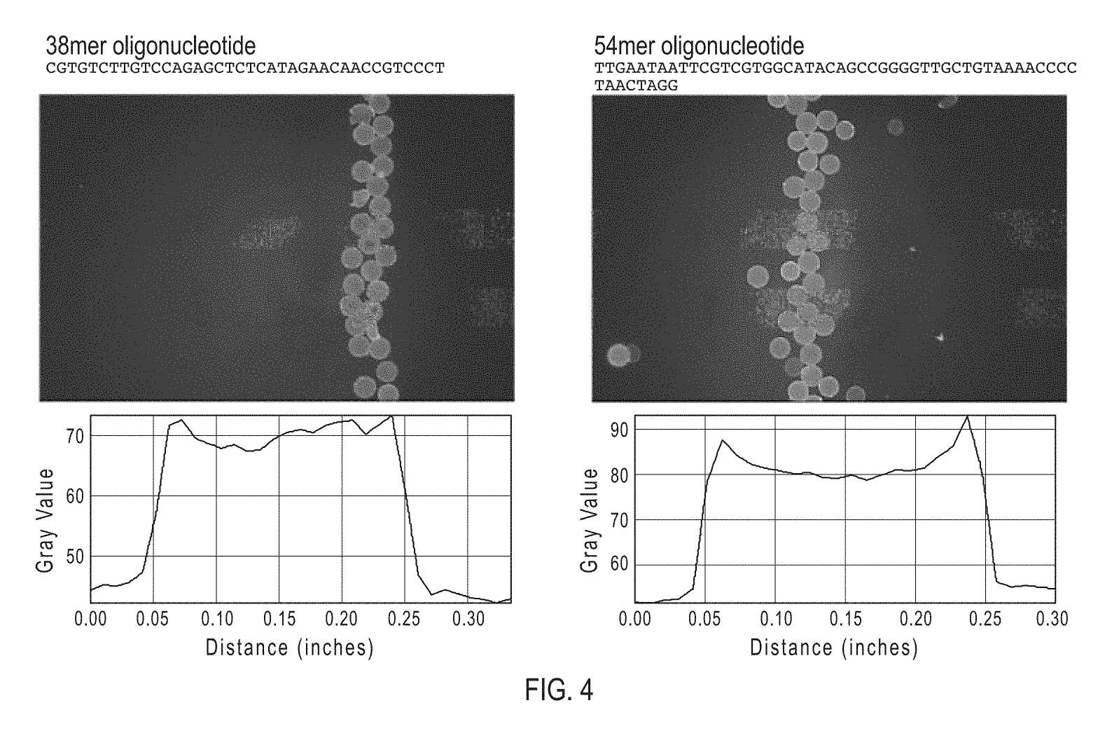

The microspheres were excited with 480 nm light and fluorescence was tracked using a Zeiss Fluorescence Microscope (see FIG. 4). The intensity of individual microspheres was plotted using ImageJ (Schneider, C. A., Rasband, W. S., Eliceiri, K. W. “NIH Image to ImageJ: 25 years of image analysis”. Nature Methods 9, 671-675, 2012). The microspheres with the 54mer oligonucleotide showed a significantly higher fluorescence intensity than the microspheres with the 38mer oligonucleotide (˜70 arbitrary fluorescence units (a.u.) VS. 85 a.u.). Since a 54mer has a greater mass than a 38mer, this result shows a semi-quantitative measurement of oligonucleotide amount.

### Example 3. Staining of Oligonucleotide Microspheres Using a Fluorogenic Dye in μ-Multiwell Plates in Hydrated State

Provided was an oligonucleotide sequence (5′-CGTGTCTTGTCCAGAGCTCTCATAGAACAACCGTCCCT-3′) (SEQ ID NO: 3), covalently bound to 30 μm polystyrene microspheres (Custom Primer Support 80S (dT, GE Healthcare)) prepared with an ABI392 DNA Synthesizer (Applied Biosystems) essentially as in Example 1.

The 30 μm polystyrene microspheres coated with fully protected oligonucleotide were dried and used directly for staining.

The microspheres were flushed into μ-multiwell plates (40 μm well depth, 40 μm well diameter, 70 μm pitch, Micronit Microfluidics) to fix the beads in an array. The microspheres were treated with acetonitrile to swell the polystyrene matrix and subsequently washed with QUANT-IT OLIGREEN® ss DNA Reagent (Thermo Fisher Scientific) in TE buffer.

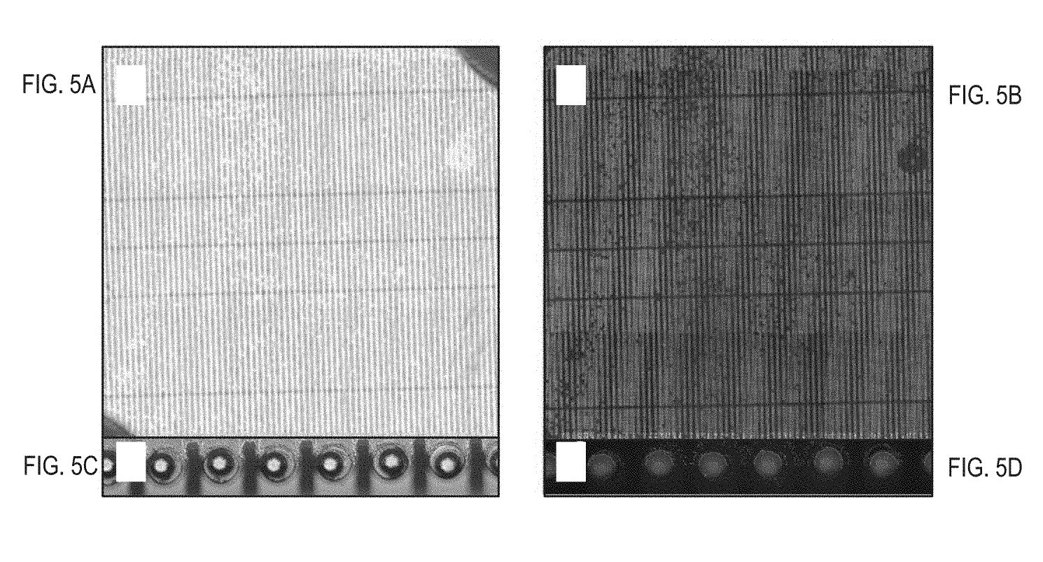

The microspheres were excited with 480 nm and fluorescence was tracked using a Zeiss Fluorescence Microscope (see FIGS. 5B & 5D). The beads showed a strong fluorescence signal.

### Example 4. Staining of Oligonucleotide Microspheres Using a Fluorogenic Dye in μ-Multiwell Plates in Dry State

An oligonucleotide-bead-array was prepared and stained with QUANT-IT OLIGREEN® as described in Example 3.

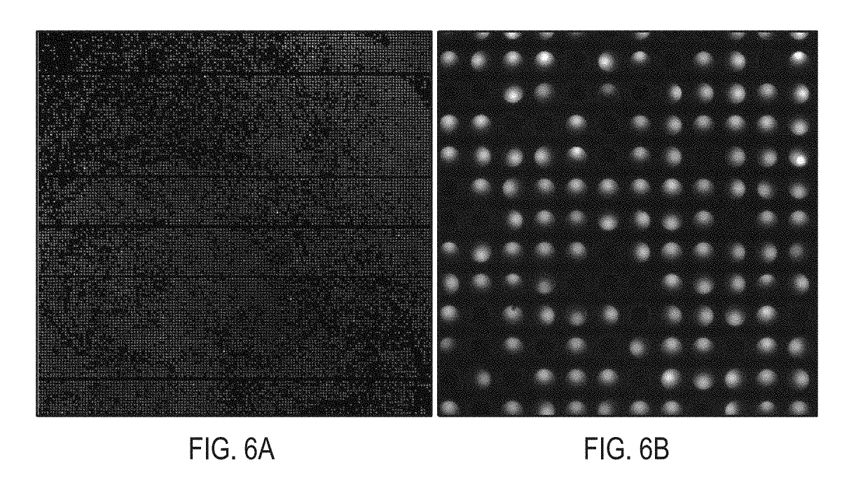

The μ-multiwell plate was dried in a vacuum centrifuge to facilitate handing of the oligo-bead-array. The μ-multiwell plate was excited with 470 nm light and fluorescence was tracked using an EVOS FL auto fluorescence microscope (see FIG. 6A-6B). The beads showed a strong fluorescence signal.

### Example 5. Removal of OLIGREEN® from Oligonucleotide Microspheres in μ-Multiwell Plates

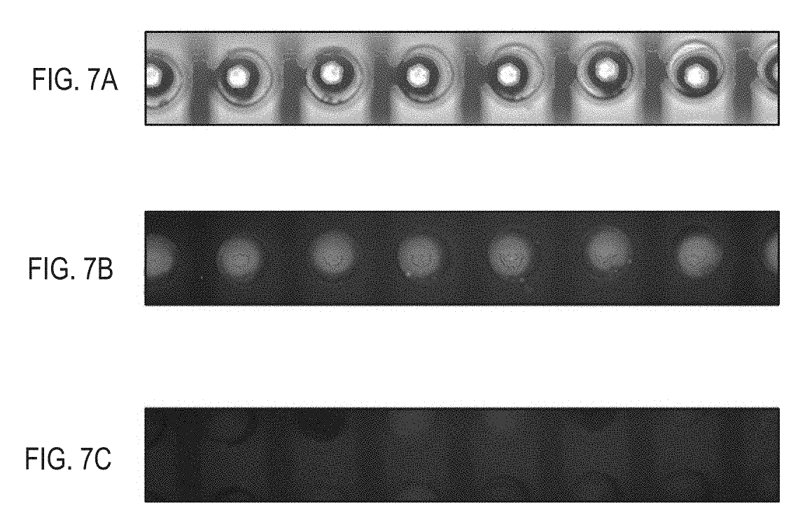

An oligonucleotide-bead-array was prepared and stained with OLIGREEN® as described in Example 3. The microspheres were excited with 480 nm light and fluorescence was tracked using a Zeiss Fluorescence Microscope (see FIG. 7A-7C). The beads showed a strong fluorescence signal before washing (see FIG. 7B).

The μ-multiwell plate was washed with a constant flow of 5 ml acetonitrile over 5 minutes. No fluorescence signal was detected after the washing step (see FIG. 7C). This demonstrated that the dye can be removed efficiently from the solid sample by a washing step.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### Exemplary Subject Matter of the Invention is Represented by the Following Clauses

Clause 1. A method of quantifying at least one nucleic acid molecule associated with a surface, the method comprising:

contacting the at least one nucleic acid molecule with a fluorophore, wherein the fluorophore emits increased fluorescence at a wavelength when in contact with nucleic acid molecule; and

measuring fluorescence from the fluorophore at the wavelength.

Clause 2. The method of clause 1, wherein the measured fluorescence is correlated to the mass of nucleic acid molecule associated with the surface.

Clause 3. The method of clause 1, wherein the measured fluorescence is correlated to the molar quantity of nucleic acid molecule associated with the surface.

Clause 4. The method of clause 3, wherein the fluorophore is attached to a specific binding agent.

Clause 5. The method of clause 4, wherein the specific binding agent comprises a nucleic acid molecule probe.

Clause 6. The method of any one of clauses 1 to 5, wherein the measured fluorescence is compared to a reference.

Clause 7. The method of clause 6, wherein the reference is a threshold value, a standard curve, or a value measured from a reference sample.

Clause 8. The method of any one of the preceding clauses, wherein the surface is a surface of a slide or chip, optionally wherein the slide or chip is a microarray or silicon wafer.

Clause 9. The method of clause 8, wherein the slide or chip comprises a semiconductor, glass, silanized glass, polyethyleneimine-coated glass, quartz, plastic, polystyrene, polypropylene, or polyethylene.

Clause 10. The method of clause 8 or 9, wherein the slide or chip is nucleophilically or electrophilically derivatized.

Clause 11. The method of any one of clauses 8 to 10, wherein the slide or chip comprises thiol, isothiocyanate, aldehyde, mercaptoalkyl, bromoacetamide, p-aminophenyl, epoxide, N-hydroxysuccinimidyl, imidoester, amino, cyanuric chloride, acrylic, carboxylic acid, maleimide, or disulfide functional groups.

Clause 12. The method of any one of clauses 8 to 11, wherein the surface is associated with a plurality of nucleic acid molecules, with the nucleic acid molecules being in discrete locations on the surface.

Clause 13. The method of any one of clauses 1 to 7, wherein the surface is the surface of a bead.

Clause 14. A method of detecting at least one nucleic acid molecule associated with a surface of a bead, the method comprising:

contacting the at least one nucleic acid molecule with a fluorophore, wherein the fluorophore emits increased fluorescence at a wavelength when in contact with a nucleic acid molecule; and

detecting fluorescence from the fluorophore at the wavelength.

Clause 15. The method of clause 14, wherein the bead is a member of a mixed population of beads.

Clause 16. The method of any one of clauses 13 to 15, wherein the bead has a size greater than or equal to about 0.05 μm, 0.1 μm, 0.2 μm, 0.3 μm, 0.5 μm, 1 μm, 2 μm, 3 μm, 5 μm, 10 μm, 20 μm, 30 μm, 40 μm, or 50 μm, and less than or equal to about 500 μm.

Clause 17. The method of any one of clauses 13 to 16, wherein the bead has a size greater than or equal to about 0.1 μm and less than or equal to about 3 μm, 5 μm, 10 μm, 20 μm, 30 μm, 40 μm, 50 μm, 100 μm, 200 μm, 300 μm, 500 μm, 1 mm, or 2 mm.

Clause 18. The method of any one of clauses 13 to 17, wherein the bead comprises plastic, ceramic, glass, polystyrene, methylstyrene, acrylic polymer, paramagnetic material, thoria sol, carbon graphite, titanium dioxide, latex, a cross-linked dextran, Sepharose, cellulose, nylon, cross-linked micelles, hydrogel, or polytetrafluoroethylene.

Clause 19. The method of any one of clauses 13 to 18, wherein the bead is suspended in an aqueous medium.

Clause 20. The method of any one of clauses 13 to 18, wherein the bead is dry.

Clause 21. The method of any one of clauses 13 to 18, wherein the bead is suspended in an organic medium.

Clause 22. The method of any one of clauses 13 to 21, wherein the bead is porous.

Clause 23. The method of any one of the preceding clauses, wherein the at least one nucleic acid molecule comprises a plurality of nucleic acid molecules with different sequences.

Clause 24. The method of any one of clauses 1 to 22, wherein the at least one nucleic acid molecule consists essentially of nucleic acid molecules with a single sequence.

Clause 25. The method of any one of clauses 1 to 24, wherein the at least one nucleic acid molecule is attached to the surface through a noncovalent interaction.

Clause 26. The method of clause 25, wherein the noncovalent interaction is between a hapten and a polypeptide or aptamer with affinity for the hapten.

Clause 27. The method of any one of clauses 1 to 24, wherein the at least one nucleic acid molecule is covalently linked to the surface.

Clause 28. The method of clause 27, wherein the at least one nucleic acid molecule was covalently linked via reaction of a disulfide, thiol, amine, carboxyl, maleimide, phosphorothioate, aldehyde, alkylamino, acrylamide, or phosphoryl on the nucleic acid molecule with the surface.

Clause 29. The method of any one of clauses 1 to 22 or 27 to 28, wherein the at least one nucleic acid molecule was synthesized in situ.

Clause 30. The method of clause 29, wherein the at least one nucleic acid molecule was synthesized in situ on a surface of a well of a multiwell plate.

Clause 31. The method of clause 29, wherein the at least one nucleic acid molecule was synthesized in situ on a surface of a bead.

Clause 32. The method of any one of clauses 29 to 31, wherein the in situ synthesis comprises enzymatic extension or ligation.

Clause 33. The method of any one of clauses 29 to 32, wherein the in situ synthesis comprises addition of phosphoramidite nucleosides.

Clause 34. The method of any one of clauses 1 to 22 or 27 to 33, wherein the method comprises synthesizing the at least one nucleic acid molecule on the surface before the contacting step.

Clause 35. The method of any one of the preceding clauses, wherein the at least one nucleic acid molecule comprises at least one nonstandard nucleotide.

Clause 36. The method of any one of the preceding clauses, wherein the at least one nucleic acid molecule comprises at least one deoxyribonucleotide.

Clause 37. The method of any one of the preceding clauses, wherein the fluorophore emits increased fluorescence at the wavelength when in contact with a single-stranded nucleic acid molecule.

Clause 38. The method of clause 37, wherein the at least one nucleic acid molecule comprises at least one single-stranded oligonucleotide.

Clause 39. The method of any one of clauses 1 to 36, wherein the fluorophore emits increased fluorescence at the wavelength when in contact with a double-stranded nucleic acid molecule.

Clause 40. The method of clause 39, wherein the at least one nucleic acid molecule comprises at least one double-stranded nucleic acid molecule.

Clause 41. The method of any one of the preceding clauses, wherein the fluorophore comprises a cyanine dye, a phenanthridinium dye, a bisbenzimide dye, a bisbenzimidazole dye, an acridine dye, a chromomycinone dye, OLIGREEN®, PICOGREEN®, SYBR® Green, SYBR® Green II, SYBR® Gold, SYBR® Safe, CYQUANT® GR, DAPI, ethidium bromide, dihydroethidium, propidium iodide, hexidium iodide, QUANTIFLUOR® ssDNA dye, QUANTIFLUOR® dsDNA dye, a benzothiazolium dye, acridine orange, proflavine HCl, thiazole orange, oxazole yellow, chromomycin A3, 7-aminoactinomycin D, hydroxystilbamidine, HOECHST® 33258, HOECHST® 33342, thiazole orange tetramethylpropane diamine, thiazole orange tetramethyl diamine, ethidium propane diamine, or ethidium diethylene triamine.

Clause 42. The method of any one of the preceding clauses, wherein the at least one nucleic acid molecule comprises at least one protected moiety.

Clause 43. The method of any one of the preceding clauses, wherein the at least one nucleic acid molecule comprises at least one unprotected nucleotide residue.

Clause 44. The method of clauses 43, wherein at least one unprotected nucleotide residue comprises an exocyclic amine.

Clause 45. The method of any one of the preceding clauses, wherein the detecting step is an in-line quality control step.

Clause 46. The method of any one of the preceding clauses, wherein, after detection, the at least one nucleic acid molecule is used in at least one downstream step.

Clause 47. The method of any one of the preceding clauses, further comprising reacting the at least one nucleic acid molecule after the detecting step.

Clause 48. The method of clauses 47, wherein the reacting comprises extending or ligating.

Clause 49. A noncovalent complex of a fluorophore and a nucleic acid molecule, wherein the nucleic acid molecule is associated with a surface of a bead, and the fluorophore has the property of emitting increased fluorescence at a wavelength when in contact with a nucleic acid molecule.

Clause 50. The complex of clause 49, wherein the fluorophore is attached to a specific binding agent.

Clause 51. The complex of clause 50, wherein the specific binding agent comprises a nucleic acid molecule probe.

Clause 52. The complex of any one of clauses 49 to 51, wherein the surface is associated with a plurality of nucleic acid molecules with different sequences.

Clause 53. The complex of any one of clauses 49 to 52, wherein the surface is associated with a plurality of nucleic acid molecules consisting essentially of nucleic acid molecules with a single sequence.

Clause 54. The complex of any one of clauses 49 to 53, wherein the at least one nucleic acid molecule is attached to the surface through a noncovalent interaction.

Clause 55. The complex of clause 54, wherein the noncovalent interaction is between a hapten and a polypeptide or aptamer with affinity for the hapten.

Clause 56. The complex of any one of clauses 49 to 53, wherein the at least one nucleic acid molecule is covalently linked to the surface.

Clause 57. The complex of clause 56, wherein the at least one nucleic acid molecule was covalently linked via reaction of a disulfide, thiol, amine, carboxyl, maleimide, phosphorothioate, aldehyde, alkylamino, acrylamide, or phosphoryl on the nucleic acid molecule with the surface.

Clause 58. The complex of any one of clauses 49 to 57, wherein the at least one nucleic acid molecule comprises at least one nonstandard nucleotide.

Clause 59. The complex of any one of clauses 49 to 58, wherein the at least one nucleic acid molecule comprises at least one deoxyribonucleotide.

Clause 60. The complex of any one of clauses 49 to 59, wherein the fluorophore emits increased fluorescence at the wavelength when in contact with a single-stranded nucleic acid molecule.

Clause 61. The complex of clause 60, wherein the at least one nucleic acid molecule comprises at least one single-stranded oligonucleotide.

Clause 62. The complex of any one of clauses 49 to 59, wherein the fluorophore emits increased fluorescence at the wavelength when in contact with a double-stranded nucleic acid molecule.

Clause 63. The complex of clause 62, wherein the at least one nucleic acid molecule comprises at least one double-stranded nucleic acid molecule.

Clause 64. The complex of any one of clauses 49 to 63, wherein the fluorophore comprises a cyanine dye, a phenanthridinium dye, a bisbenzimide dye, a bisbenzimidazole dye, an acridine dye, a chromomycinone dye, OLIGREEN®, PICOGREEN®, SYBR® Green, SYBR® Green II, SYBR® Gold, SYBR® Safe, CYQUANT® GR, DAPI, ethidium bromide, dihydroethidium, propidium iodide, hexidium iodide, QUANTIFLUOR® ssDNA dye, QUANTIFLUOR® dsDNA dye, a benzothiazolium dye, acridine orange, proflavine HCl, thiazole orange, oxazole yellow, chromomycin A3, 7-aminoactinomycin D, hydroxystilbamidine, HOECHST® 33258, HOECHST® 33342, thiazole orange tetramethylpropane diamine, thiazole orange tetramethyl diamine, ethidium propane diamine, or ethidium diethylene triamine.

Clause 65. The complex of any one of clauses 49 to 64, wherein the at least one nucleic acid molecule comprises at least one protected moiety.

Clause 66. The complex of any one of clauses 49 to 65, wherein the at least one nucleic acid molecule comprises at least one unprotected nucleotide residue.

Clause 67. The complex of clauses 66, wherein at least one unprotected nucleotide residue comprises an exocyclic amine.

Clause 68. The complex of any one of clauses 49 to 67, wherein the bead has a size greater than or equal to about 0.05 μm, 0.1 μm, 0.2 μm, 0.3 μm, 0.5 μm, 1 μm, 2 μm, 3 μm, 5 μm, 10 μm, 20 μm, 30 μm, 40 μm, or 50 μm, and less than or equal to about 500 μm.

Clause 69. The complex of any one of clauses 49 to 68, wherein the bead has a size greater than or equal to about 0.1 μm and less than or equal to about 3 μm, 5 μm, 10 μm, 20 μm, 30 μm, 40 μm, 50 μm, 100 μm, 200 μm, 300 μm, 500 μm, 1 mm, or 2 mm.

Clause 70. The complex of any one of clauses 49 to 69, wherein the bead comprises plastic, ceramic, glass, polystyrene, methylstyrene, acrylic polymer, paramagnetic material, thoria sol, carbon graphite, titanium dioxide, latex, a cross-linked dextran, Sepharose, cellulose, nylon, cross-linked micelles, hydrogel, or polytetrafluoroethylene.

Clause 71. The complex of any one of clauses 49 to 70, wherein the bead is suspended in an aqueous medium.

Clause 72. The complex of any one of clauses 49 to 70, wherein the bead is dry.

Clause 73. The complex of any one of clauses 49 to 70, wherein the bead is suspended in an organic medium.

Clause 74. The complex of any one of clauses 49 to 73, wherein the bead is porous.

Clause 75. The complex of any one of clauses 49 to 74, for use in detecting or quantifying the nucleic acid molecule.

Clause 76. An apparatus comprising a fluorescence excitation source, a fluorescence detector, and the complex of any of clauses 49 to 74.

1. A method of quantifying at least one nucleic acid molecule associated with a surface, the method comprising:
contacting the at least one nucleic acid molecule with a fluorophore, wherein the fluorophore emits increased fluorescence at a wavelength when in contact with nucleic acid molecule; and
measuring fluorescence from the fluorophore at the wavelength.

2.-3. (canceled)

4. The method of claim 1, wherein the fluorophore is attached to a specific binding agent.

5. The method of claim 4, wherein the specific binding agent comprises a nucleic acid molecule probe.

6. The method of claim 1, wherein the measured fluorescence is compared to a reference.

7. The method of claim 6, wherein the reference is a threshold value, a standard curve, or a value measured from a reference sample.

8.-12. (canceled)

13. The method of claim 1, wherein the surface is the surface of a bead.

14. A method of detecting at least one nucleic acid molecule associated with a surface of a bead, the method comprising:
contacting the at least one nucleic acid molecule with a fluorophore, wherein the fluorophore emits increased fluorescence at a wavelength when in contact with a nucleic acid molecule; and
detecting fluorescence from the fluorophore at the wavelength.

15. The method of claim 14, wherein the bead is a member of a mixed population of beads.

16. (canceled)

17. The method of claim 14, wherein the bead has a size ranging from 5 μm to 100 μm.

18. (canceled)

19. The method of claim 14, wherein the bead is suspended in an aqueous medium.

20.-21. (canceled)

22. The method of claim 14, wherein the bead is porous.

23.-26. (canceled)

27. The method of claim 1, wherein the at least one nucleic acid molecule is covalently linked to the surface.

28.-46. (canceled)

47. The method of claim 14, further comprising reacting the at least one nucleic acid molecule after the detecting step.

48. The method of claim 47, wherein the reacting comprises extending or ligating.

49. A noncovalent complex of a fluorophore and a nucleic acid molecule, wherein the nucleic acid molecule is associated with a surface of a bead, and the fluorophore has the property of emitting increased fluorescence at a wavelength when in contact with a nucleic acid molecule.

50. The complex of claim 49, wherein the fluorophore is attached to a specific binding agent.

51. The complex of claim 50, wherein the specific binding agent comprises a nucleic acid molecule probe.

52. The complex of claim 49, wherein the surface is associated with a plurality of nucleic acid molecules with different sequences.

53.-67. (canceled)

68. The complex of claim 49, wherein the bead has a size ranging from 5 μm to 100 μm.

69.-75. (canceled)

76. An apparatus comprising a fluorescence excitation source, a fluorescence detector, and the complex of claim 49.
